(19) United States
(12) Patent Application Publication
(10) Pub. No.: US 2017/0032080 A1
(43) Pub. Date: Feb. 2, 2017

(54) Method and arrangement for matching mammals by comparing genotypes

(71) Applicant: Genoscoper OY, Helsinki (FI)

(72) Inventors: Hannes Lohi, Vantaa (FI); Tuomas Poskiparta, Espoo (FI)

(73) Assignee: Genoscoper, Helsinki (FI)

(21) Appl. No.: 15/106,366

(22) PCT Filed: Dec. 18, 2014
(86) PCT No.: PCT/FI2014/051029

§ 371 Date: Jun. 20, 2016

(30) Foreign Application Priority Data
Dec. 19, 2013 (FI) 20136293

(51) Int. Cl. G06F 19/18; G06F 19/22; G06F 19/28; A01K 29/00; A01K 67/02

(52) U.S. Cl. CPC G06F 19/18; A01K 29/00; A01K 67/02; G06F 19/28; G06F 19/22

(57) ABSTRACT

A method for determining a matchmaking quality for a first mammal in relation to a plurality of different second mammals by comparing genotypes thereof, comprises steps of providing a first database and second database. The first database comprises genotyping data of known traits and coefficients weighting severity of said traits, and the second comprises genotyping data for at least certain loci common to each of said mammals to be matchmade. In determination of value of the match first results of the coefficients weighting the severities of the traits are determined for each potential breeding pair of the first mammal with each of said plurality of the different second mammals, and second results relating to a diversity are determined for each potential breeding pair of the first mammal with each of said plurality of the different second mammals. A total result determining the matchmaking quality for each potential breeding pair is determined by combining said first and second results for each of said potential breeding pair.

### TECHNICAL FIELD OF THE INVENTION

The invention relates to a method and arrangement for determining a matchmaking and offering a proposal for match of a first mammal in relation to a plurality of different second mammals. In particularly the invention relates to analysing genomic data of the first and plurality of second mammals in order to achieve probability or severity of different traits and/or genetic diversity of the possible descendants of the first mammals with potential second mammals, such as disease, morphology and/or behaviour traits.

### BACKGROUND OF THE INVENTION

Gene discoveries and understanding of the genomic structures of the mammals enable different types of genetic tests for breeding purposes. For example, a DNA test can discriminate genetically normal, carrier and affected mammals from each other and help breeders to improve breeding plans to avoid affected puppies caused by a known disease mutation. Veterinarians can use tests as diagnostic tools. Systematic and careful use of the DNA tests may help to reduce the incidence of the diseases in the breed or even eradicate them from the populations while maintaining necessary genetic diversity given that unaffected mutation carriers can be kept in the breeding programs. Genetic diversity can also be maintained by avoiding inbreeding, e.g. use of close relatives in the breeding programs utilizing for example existing pedigree databases. However, pedigrees are often complex in the closed breeding populations such as the dog breeds, and pedigree analyses may give inaccurate estimates about relatedness. Animals may be more related to each other than expected based on pedigrees. For a better evaluation, direct measurement of the genetic diversity by a DNA test with a reasonable density of markers for an efficient coverage of the genome is often recommended. The maintenance and development of the genetic diversity of the populations is very important for example in dog, cat and horse breeding, but also with more rare breeds, such as of llama, camel or zebra.

Genetic traits can be inherited in many ways. A common mode of inheritance in inbred populations is autosomal recessive, although some autosomal dominant and X-linked traits exist. These so called Mendelian traits cause usually single gene disorders. However, it is important to keep in mind that the penetrance of the disease may vary, and the phenotypic expression of the condition in individuals with the same mutation may differ. Many common disorders are polygenic affected by several genes and environmental factors. Several genetic loci contribute to the disease risk and the onset and outcome of disease is defined by the combination of risk genes and environmental factors.

Public annotations of domestic animal genomes including dogs and horses and subsequent development of genomic tools for gene mapping greatly facilitate gene discoveries for disease, conformation, behaviour and performance Furthermore, the identification of millions of genomic variants in each genome enables the development of tools to measure genetic diversity and ancestry. Simultaneous rapid development of economic high-resolution sequencing and genotyping technologies revolutionizes DNA diagnostics and transforms the field from the analysis of single genetic regions to the interpretation of the entire genomes of individual animals. This type of extensive genome wide information allows combined analysis of various features of the tested mammal, including information such as ancestry or parentage, genetic diversity, multiple disease, morphological and behavioural traits. However, the analysis or interpretation of the genomic data requires prior information about the correlation of a specific marker or markers with particular phenotype or phenotypes with efficient bioinformatics methods. In addition, the plurality of the results generated by a genome wide analysis requires the development of new reporting tools for better and sufficient understanding of the scientific data also to those who do not have expertise or experience in such information, including owners, breeders and veterinarians.

Although genotyping and new next generation sequencing (NGS) technologies allow genome wide analyses of individual animals, there are disadvantages in the generation and interpretation of the genomic data. The challenges are related to the technical quality and reliability of the NGS data, to the large amount, mining and storage of the data for bioinformatics interpretation and to the expensive cost of the laboratory experiments. Thus in order to test or determine plurality of trait of the mammal takes time and is therefore quite expensive.

The other disadvantages include the lack of proper methods for existing trait correlations that makes the interpretation of the data very slow and complicated. In addition, the known trait-specific correlated DNA markers can be of many different types such as single nucleotide polymorphism (SNP), microsatellite (di- or tetranucleotide repeats), indels, block substitutions, inversions or copy number variant (CNV). Currently, there has not been a single reliable cost efficient technology that could read or sequence or genotype all different types of markers simultaneously from targeted regions of the genome of a tested animal for a comprehensive genetic analysis of the animal's ancestry, health risk, morphology and behaviour, and especially for breeding purposes.

### SUMMARY OF THE INVENTION

An object of the invention is to alleviate and eliminate the problems relating to the known prior art. Especially the object of the invention is to provide a method and an arrangement or system for determining a match for a seed mammal with a potential partner mammal selected from the group of second mammals so that their offsprings would be genetically more diverse and do not carry or become affected for at least certain traits or diseases determined beforehand. In particularly the object of the invention is to identify genetically most appropriate match for the seed mammals among the plurality of second mammals and propose the found partner of the second mammals for the breeding purpose.

The object of the invention can be achieved by the features of independent claims.

The invention relates to a method for determining a matchmaking for a first mammal in relation to a plurality of different second mammals by comparing genotypes thereof according to claim 1. In addition the invention relates to a corresponding arrangement according to claim 13, as well as to a computer program of claim 16.

According to an embodiment a first database is provided, where said database comprises clinical and genetic data of known traits and coefficients weighting severity of said traits, such as details of mutation, disease risk, onset, lethality and/or affected breeds of at least one mammal species and especially of mammal species of said mammals to be matchmade. The data of the known traits is advantageously based on the scientific research and commonly known information. The coefficients weighting severity of said traits is used here for a specific genomic variation that has been associated with or shown to modulate or affect the disease, morphology or behaviour, but it may also be a statistical association or is often supported by functional evidence. These coefficients are based advantageously on experimental clinical data, which is, according to an example, defined based on the published descriptions of the traits. As an example the coefficients are numerical data, such as a number indicating relational severity of a certain trait in relation to another trait.

Said data in the first database advantageously relates to scientific research, which identifies new genes for example for different canine traits including disease, morphology and behaviour. Identified correlations or coefficients are provided in said first database including the details of the mutation, disease risk and the affected breeds. The first database may also comprise a compiled literature of the known correlations in various traits to be included in a so called bundle gene test. According to an example the first database comprises data of known trait loci (specific location of a gene or DNA sequence on a chromosome) and neutral loci used to measure other genetic characteristics such as genetic diversity and ancestry. These loci are determined by markers, which are to be tested for the mammals to be matchmade and in order to provide appropriate match.

In addition, a second data database is provided to comprise genotyping data for at least certain loci common to each of said individual mammals to be matchmade. According to an example the genotyping data of said individual mammals is gathered via DNA testing and analysis, which can be achieved for example from blood or a cheek swab samples. The second database may thus comprise analysed genomic data, i.e. genotyping data of said individual mammals to be matchmade.

According to an invention, a group of markers is provided, where each of said markers relates to a certain loci or locus of the genotyping information to be determined for matchmaking at least two mammals (the first and at least one of the second mammals). The group of markers is advantageously divided to at least two portions, where at least first portion of the markers relates to loci to be used for determining health related traits, and at least second portion of the markers (differing from said first portion) is used for determining genetic diversity of at least two mammals to be matchmade.

Genotypes of the loci of the mammals to be matchmade, where the loci are determined by said first portion of the markers, are then analysed and combined for possible breeding of said mammals in said the second database, where possible result of combinations (breed) is achieved by principles of known genetics. The used markers relate also to corresponding loci in the first database, disease risks, morphology and/or behaviour) for possible breeding combinations derived in said second database.

According to an embodiment to identify genetically appropriate match partner amongst the plurality of potential different second mammals for said seed (first) mammal, potential partners are sorted or ranked into an order describing their genomic health quality of different traits as well as the level of heterozygosity and genetic diversity. This method comprises to determine a first set of results of the coefficients weighting the severities of the traits for each potential partner pairs of the seed (first) mammal with each of said plurality of the different potential partner mammals (so called second mammals) by using said first portion of the markers. This is advantageously to provide a trait severity load index (advantageously numerical data) for the expected offspring. As an example, the total severity load for an individual mammal can be calculated as the sum of all genotypic risk weighted by the severity of the conditions, but also other mathematical functions can be used. In addition, the method comprises to determine also second results relating to the genetic diversity for each potential partner pairs of the seed (first) mammal with each of said plurality of the different potential partner mammals (so called second mammals) by using said second portion of the markers. This is advantageously to evaluate the possible genotype composition and frequency at each investigated loci in a predicted group of offspring.

Furthermore, a total result describing the matchmaking quality for each potential couple (seed mammal+potential partner amongst the plurality of second mammals) is determined by combining (can be calculated as the sum, for example) said first and second results for each of said potential partner pairs for example into a combined index reflecting disease load and diversity. The primary match partner amongst the plurality of the second mammals for the seed mammal (first mammal) is then determined by sorting or ranking the total results for each of said potential partner pair into a preferential order desired, like the most point earner partner of the second mammals is placed at the most top in the order.

In addition, according to another embodiment also a third database is provided including phenotyping data of the mammals (second mammal) to be matchmade. The phenotyping data relates advantageously to known phenotyping traits such as morphology, behaviour, preferred use of the mammal (for example a hunting dog, companion dog, agility dog or service dog), color, health conditions including for example hip dysplasia status or eye exam, temperament, showing results, hunting skills of mammal species of said mammals to be matchmade. Still, according to an embodiment said third database may comprise coefficients weighting severity or probability or other weighting of said phenotyping traits such as owner-completed questionnaire data or points or ranks in the field, show or temperament tests as defined by the external judges according to set rules for such situations. Phenotyping data in the third database can be used to define desired features from the mammals to be matchmade or filter out mammals with undesired phenotypic features. The selection of such phenotypic seed may affect the ranking of the possible breeding partners. For example, the genetically most appropriate partner may drop in the ranking because of lacking desired phenotypic features, such as sufficient success in the hunting test, desired by the breeder.

According to an exemplary embodiment at least one phenotype is selected before matching as a phenotype seed character, like for example use of the mammal or color. The phenotype seed character may be e.g. a number, which might be weighted by the desired severity, which must be achieved at least if the first and second mammals are bred.

According to an example the phenotype seed may relate to at least one phenotype to be desired or avoided for the potential descendants. In the example a loci related to said phenotype in the first database is analysed for both first seed mammal and at least one of the second mammal to be matchmade in said second database and then finally the corresponding coefficients weighting severity of said phenotyping trait is selected in said third database. If said seed is the desired phenotype and said coefficients is over (>) a threshold, the partners matchmade is valid for breeding for said phenotyping traits. However, if said seed is the undesired phenotype and said coefficients over (>) a threshold, the partners matchmade is invalid for breeding for said phenotyping traits. If several second mammals fullfill the minimal threshold criteria of the set desired traits, they will be ranked according to genetic appropriateness.

According to an example the phenotyping matchmade may be required to be done first before any genotyping based matchmade, whereupon only potential breeding partners having a certain probability over a predetermined threshold for the desired phenotype in their breed are selected to the determination of the genotyping based matchmade.

Still according to an embodiment at least one genotyping or phenotyping data referred by the markers (relating to e.g. a certain trait, like disease or character) is and thereby producing invalid breeding value for said two mammals to be matchmade, if their genotype or phenotype matches with said genotyping or phenotyping data referred by said markers.

The phenotype data in the third database can be achieved e.g. so that phenotypic profiles for the mammals can be filled out e.g. via data processing systems. The system may offer an opportunity to participate for example in scientific studies with more in-depth surveys. According to an embodiment new correlations between the genotypic data and phenotypic data may also be revealed by comparing portions of said two different data with each other, such as to define new genetic correlations, to provide large study cohorts to academic research groups, to partnership with mammal food and pharmacy industries for the development of better products or to improve the fidelity of the existing ones.

According to an example the first database may also comprise neutral genotyping data used only for diversity determination in addition to said second portion of said markers, or also neutral phenotyping data such as for example pedigrees is used for diversity determination.

As an example the first portion of the markers used may comprise over 50 markers, more advantageously over 100 markers and most advantageously over about 150 markers, the majority of which are advantageously disease related markers or markers associated with morphological, such as conformation, colour, fur type, hair length, or behavioural traits of the animal. In addition, as an example, the second portion of said markers may comprise advantageously over 200 neutral markers, more advantageously over about 500 markers and most advantageously over about 1000 markers, relating to for example microsatellite- and/or SNP-markers.

Marker means according to an exemplary embodiment a loci or locus of a known trait (e.g. disease, morphology, behaviour)-causing mutation (SNP, indel, CNV) or associated risk marker (SNP) or neutral diversity marker (microsatellite/SNP).

After analysing and determination the matchmaking of the mammals using at least two of the databases with plurality of the traits and markers, the results may be reported e.g. visually via a graphical interface and/or via simplified listed numerical data. Said results may be reported or visualised for example by using fourfold table, or Gaussian curve so that one can see e.g. health risks for different breeding pairs in one go or at a glance.

The invention offers many advantages over the known prior art, such as an efficient tool to combine plurality of complex genetic and phenotypic data to find genetically and phenotypically appropriate candidate matches for mammals for breeding purposes. The need for such as tool is highly warranted given the rapid rate of new gene and variant discoveries for diseases, conformation, performance, ancestry and genetic diversity (through accumulation of samples as well as phenotype and genotype information). In addition, the invention allows an easy and rapid way to analyse and interpret the genetic structures of the breeds and identify potential breeding partners. The invention also enables parallel analysis of genomic variants for multiple traits and provides i) more holistic genomic tool for owners and breeders to understand their animals for breeding purposes and to advance the health and welfare of the animals, ii) more comprehensive tool for veterinarians to improve diagnostics and iii) a much-needed comprehensive tool for breed clubs and associations to follow the development of the genetic diversity within and across breeds and species. Especially the invention allows an easy determination of probabilities or severities of different diseases (e.g. certain eye diseases), a simultaneous prediction of disease risk for various traits based on the genotype data for the potential descendants, and to distinct carriers and non-carriers in the breed to improve breeding decisions to eliminate disease from the breed, as well as to keep healthy carriers in breeding programs to maintain genetic diversity.

In addition it is to be noted that the first database or so called literature database of the invention compiles the list and interpretation of the latest gene tests and is very useful for veterinarians, academic and animal community by providing information on trait correlations and related risks in a single website.

### BRIEF DESCRIPTION OF THE DRAWINGS

Next the invention will be described in greater detail with reference to exemplary embodiments in accordance with the accompanying drawings, in which:

FIG. 1 illustrates a principle of an exemplary method for determining an advised breeding choice or a matchmaking quality according to an advantageous embodiment of the invention, and

FIGS. 2A-2B illustrate principles of two exemplary methods for determining an advised breeding choice or a matchmaking quality according to an advantageous embodiment of the invention.

### DETAILED DESCRIPTION

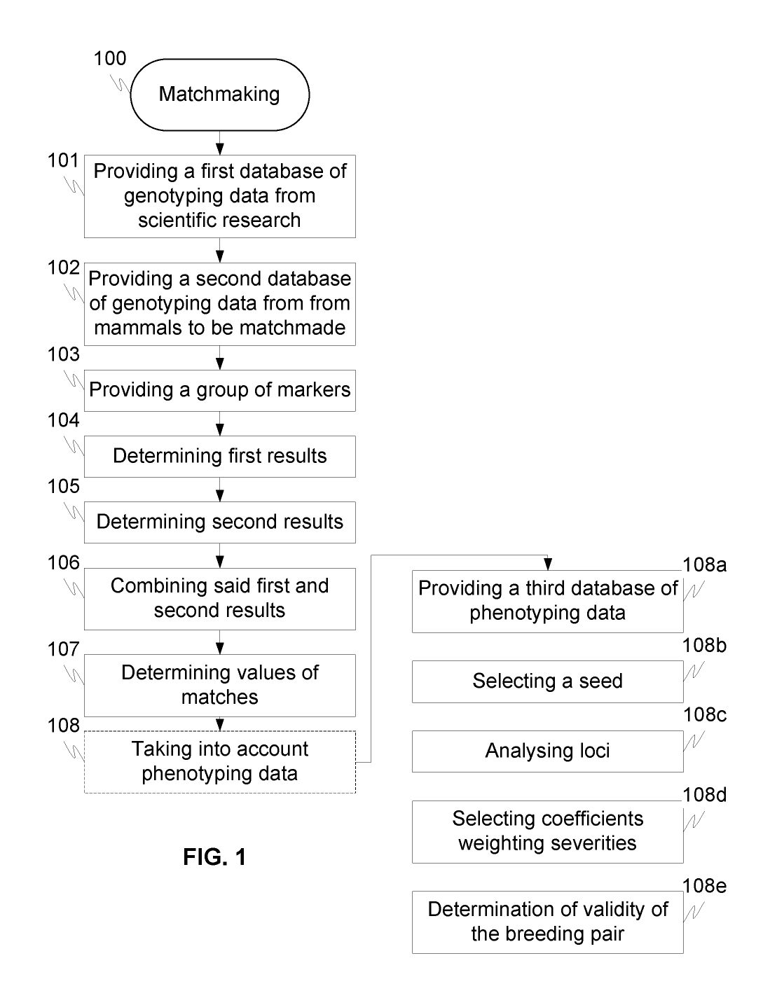

FIG. 1 illustrates a principle of an exemplary method 100 for determining an advised breeding choice or a matchmaking quality for a first mammal in relation to a plurality of different second mammals by comparing genotypes thereof according to an advantageous embodiment of the invention. In step 101 a first database is provided. The first database comprises genotyping data of known traits and coefficients weighting severity of said traits, such as mutation, disease risk, lethality and/or affected breeds of mammal species of said mammals to be matchmade. In second step 102 a second database is provided. The second database comprises genotyping data for at least certain loci common to each of said mammals to be matchmade, where said genotyping data is advantageously gathered via testing from the mammals to be matchmade.

In addition in step 103 a group of markers is provided for the loci to be determined for matchmaking. At least first portion used for determining health related traits of the markers relates to loci to be compared in the second databases and having the coefficients in various known gene based traits in said first databases for possible breeding combinations derived in said second database. At least second portion of said markers used in determining differs from said first portion of said markers.

Next value of the match for said first mammal with said plurality of the different second mammals is determined. In step 104 first results of said coefficients weighting the severities of the traits are determined for each potential breeding pair of the first mammal with each of said plurality of the different second mammals by using said first portion of the markers. In step 105 second results relating to a diversity are determined for each potential breeding pair of the first mammal with each of said plurality of the different second mammals by using said second portion of the markers.

Finally in step 106 a total result determining the matchmaking quality for each potential breeding pair is determined by combining said first and second results for each of said potential breeding pair. In addition in step 107 said total results for each of said potential breeding pairs are manipulated in order to determine values of matches for said first mammal. The manipulation may be for example sorting or ranking the total result into a certain order and thereby determining values of matches, such as the best match.

It is to be noted that the method 100 may comprise also additional step 108 for taking into account also phenotyping data. The step 108 comprises providing 108a a third database, where the third database comprises phenotyping data of known phenotyping traits and coefficients weighting severity of said phenotyping traits, such as morphology, behaviour, colour, temperament, hunting skills of mammal species of said mammals to be matchmade, for at least certain loci common to each of said mammals to be matchmade. The step 108 advantageously comprises selecting 108b at least one phenotype as a seed. The seed relates to at least one phenotype to be desired or avoided for the potential descendants. The seed may be one number, weighted by the desired severity, or averaged from the number of desired or undesired phenotyping traits, for example.

The step 108 additionally and advantageously comprises also analysing 108c the loci related to said phenotype for both first mammal and said at least one of the second mammal to be matchmade in said second database. In addition it comprises selecting 108d of the corresponding coefficients weighting severity of said phenotyping trait in said third database. Furthermore the step 108 comprises determination 108e of validity of the breeding pair. This may include e.g. following comparison:
if the seed is the desired phenotype and said coefficients>a threshold, the breeding pair matchmade is valid for breeding for said phenotyping traits, and
if said seed is the undesired phenotype and said coefficients>a threshold, the breeding pair matchmade is invalid for breeding for said phenotyping traits.

It is to be noted that step 108 is optional and can be performed in any step. Especially it is to be noted that it can be performed before, during or after the step 104-107.

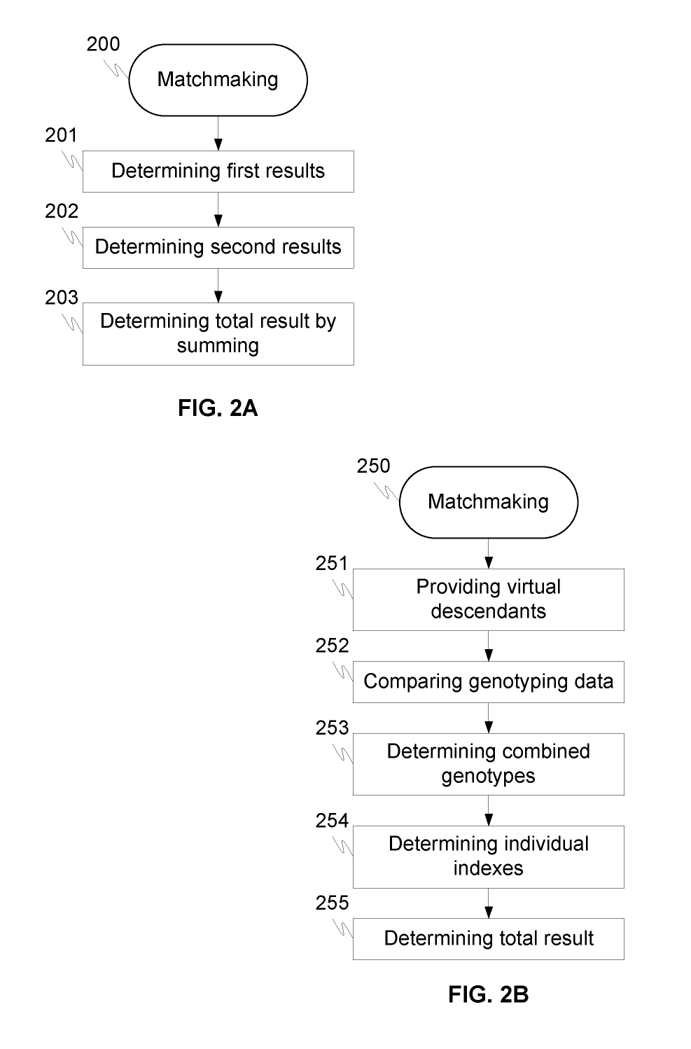

FIGS. 2A-2B illustrate principles of two exemplary methods 200, 250 for determining an advised breeding choice or a matchmaking quality according to an advantageous embodiment of the invention, where both examples are based on 103 of providing first and second databases as well as group of markers are also comprised by the methods 200, 250. In addition also step 108 with its sub-steps 108a-108e can be included to the methods 200, 250.

In the first exemplary method 200 first results of said coefficients weighting the severities of the traits are determined in step 201 for the potential breeding partners of the first mammal with each of said plurality of the different second mammals by using said first portion of the markers. In the method a sub-result of the first results relates to a certain genotype, in particularly a genotype of a locus, pointed by one of the first marker and has a certain sub-coefficient, whereupon the first results are advantageously determined in step 201 by summing said sub-coefficients of said sub-results.

In addition second results relating to the diversity are determined in step 202 by using said second portion of the markers (e.g. similarly than in step 106 in FIG. 1). If genotype of a certain locus is different for said first mammal than for the potential partner of said different second mammals in the second database, said sub-coefficient for said locus is rewarded. Rewarding may be done e.g. by giving e.g. “1”, as an example. If the genotype of said locus is same for both mammals, said sub-coefficient for said locus is not awarded or is penalized, for example by giving “0”, for example. The second results are determined advantageously by summing said sub-coefficients.

The total result is determined in step 203 by summing said first and second results.

In the second exemplary method 250 a group of virtual descendants is provided, such as created, in step 251 for different combinations of said first and at least one of said second different mammals to be matchmade. According to an example e.g. 512 descendants is provided. In step 252 genotyping data relating advantageously to at least the first portion of the markers of the first mammal's genome is compared with corresponding genotyping data of second different mammal's genome in the second database.

Next possible combined genotypes are determined in step 253 based on said compared genotyping data for each of virtual descendants to be provided. In step 254 an individual index is determined for health related traits and/or genetic diversity for each of said virtual descendants to determine the value of disease risks of said descendants and breeding value of said two mammals to be matchmade. Determination includes comparing the genotyping data of each of said individual virtual descendants' genome corresponding to said first portion of said markers with corresponding genotyping data of said first database in order to determine coefficients of the first results between said determined regions and data of said first database and thereby determine said individual index for each of said individual virtual descendants of a probability or risk or severity of the traits. The first and second results are combined (as in step 106 in FIG. 1) to determine said individual index of said virtual descendants and breeding value of said two mammals to be matchmade as said total result (step 255). The combination of said first and second results may be e.g. combination of averages of said first results and second results, as an example.

The methods may also advantageously comprise additional step (not shown) for determining matchmaking quality and the breeding value. The step of determining matchmaking quality and the breeding value may comprise steps of comparison, where:
if said individual index<a threshold, the partners matchmade is invalid for breeding,
if said individual index=a threshold, the partners matchmade is invalid for breeding, and
if said individual index>a threshold, the partners matchmade is valid for breeding.

The invention has been explained above with reference to the aforementioned embodiments, and several advantages of the invention have been demonstrated. It is clear that the invention is not only restricted to these embodiments, but comprises all possible embodiments within the spirit and scope of the inventive thought and the following patent claims. It is to be noticed that even though different databases are discussed above, they can also be implemented by a same physical database arrangement for example by allocating separated data structures for different types of data. In addition even if only few mammals are discussed as an example, the invention is not limited only to those but can be used in connection with different kinds of breeds and mammals.

1. A method for determining an advised breeding choice or a matchmaking quality for a first mammal in relation to a plurality of different second mammals by comparing genotypes thereof, wherein the method comprises steps of:
providing a first database comprising genotyping data of known traits and coefficients weighting severity of said traits, such as mutation, disease risk, lethality and/or affected breeds of mammal species of said mammals to be matchmade,
providing a second database comprising genotyping data for at least certain loci common to each of said mammals to be matchmade,
providing a group of markers for said loci to be determined for matchmaking, where
i) at least first portion of the markers relates to loci to be compared in said second databases and having the coefficients in various known gene based traits in said first databases for possible breeding combinations derived in said second database, and
ii) at least second portion of said markers used in determining differs from said first portion of said markers,
wherein in determination of value of the match for said first mammal with said plurality of the different second mammals:
first results of said coefficients weighting the severities of the traits are determined for each potential breeding pair of the first mammal with each of said plurality of the different second mammals by using said first portion of the markers, and
second results relating to a diversity are determined for each potential breeding pair of the first mammal with each of said plurality of the different second mammals by using said second portion of the markers,
a total result determining the matchmaking quality for each potential breeding pair is determined by combining said first and second results for each of said potential breeding pair, and
said total results for each of said potential breeding pairs are processed in order to determine values of matches for said first mammal.

2. A method of claim 1 for determining said matchmaking quality, wherein
first results of said coefficients weighting the severities of the traits are determined for the potential breeding partners of the first mammal with each of said plurality of the different second mammals by using said first portion of the markers, where a sub-result of the first results relates to a certain genotype and has a certain sub-coefficient, whereupon the first results are determined by summing said sub-coefficients of said sub-results, and second results relating to the diversity are determined by using said second portion of the markers so that if genotype of a certain locus is different for said first mammal than for the potential partner of said different second mammals in the second database, said sub-coefficient for said locus is rewarded and if genotype of said locus is same for both mammals, said sub-coefficient for said locus is not awarded or is penalized, whereupon the second results are determined by summing said sub-coefficients, whereupon said total result is determined by summing said first and second results.

3. The method of claim 1 for determining matchmaking quality, wherein a group of virtual descendants is provided for different combinations of said first and at least one of said second different mammals to be matchmade, where
i) genotyping data of the first mammal's genome is compared with corresponding genotyping data of second different mammal's genome in the second database,
ii) possible combined genotypes is determined based on said compared genotyping data for each of virtual descendants to be provided,
iii) an individual index is determined for health related traits and/or genetic diversity for each of said virtual descendants to determine the value of disease risks of said descendants and breeding value of said two mammals to be matchmade, comprising the steps of:
a) comparing the genotyping data of each of said individual virtual descendants' genome corresponding to said first portion of said markers with corresponding genotyping data of said first database in order to determine coefficients of the first results between said determined regions and data of said first database and thereby determine said individual index for each of said individual virtual descendants of a probability or risk or severity of the traits, and
b) combining said first results and second results, such as averages of said first results and second results to determine said individual index of said virtual descendants and breeding value of said two mammals to be matchmade as said total result.

4. The method of claim 3, wherein the matchmaking quality and the breeding value is determined as:
i) if said individual index<a threshold, the partners matchmade is invalid for breeding,
ii) if said individual index=a threshold, the partners matchmade is invalid for breeding, and
iii) if said individual index>a threshold, the partners matchmade is valid for breeding.

5. The method of claim 1, wherein a third database is provided for comprising phenotyping data of known phenotyping traits and coefficients weighting severity of said phenotyping traits, such as morphology, behaviour, color, temperament, hunting skills of mammal species of said mammals to be matchmade, for at least certain loci common to each of said mammals to be matchmade, whereupon at least one phenotype is selected as a seed.

6. The method of claim 5, wherein said seed relates to at least one phenotype to be desired or avoided for the potential descendants, whereupon:
said loci related to said phenotype is analysed for both first mammal and said at least one of the second mammal to be matchmade in said second database and the corresponding coefficients weighting severity of said phenotyping trait is selected in said third database, and,
if said seed is the desired phenotype and said coefficients>a threshold, the couple matchmade is valid for breeding for said phenotyping traits, and
if said seed is the undesired phenotype and said coefficients>a threshold, the couple matchmade is invalid for breeding for said phenotyping traits.

7. The method of claim 1, wherein said first database comprises also neutral genotyping data used only for diversity determination in addition to said second portion of said markers, and/or wherein the method uses also neutral phenotyping data such as pedigrees for diversity determination.

8. The method of claim 1, wherein at least one genotyping data of the marker relating to a certain trait is defined as overruling all other genotyping data to be determined and thereby producing invalid breeding value for said two mammals to be matchmade, such as a carrier genotype for a lethal disease in both of the breeding partners, therefore, potentially producing unviable offsprings.

9. The method of claim 1, wherein the method comprises reporting said probability or severity of the traits of said mammal as well as DNA profile via a graphical interface and/or via simplified numerical data.

10. The method of claim 1, wherein the first portion of said markers comprises over 50 markers, more advantageously over 100 markers and most advantageously over about 150 markers, the majority of which are advantageously disease markers or markers associated with morphological, such as conformation, colour, fur type, hair length, or behavioural traits of the animal.

11. The method of claim 1, wherein the second portion of said markers comprises advantageously over 200 markers, more advantageously over about 500 markers and most advantageously over about 1000 markers, relating to microsatellite- and/or SNP-markers used for the measurement of genetic ancestry and diversity.

12. The method of claim 1, wherein genomic data for a gene test is achieved to the second database by a blood or cheek swab samples.

13. An arrangement for determining an advised breeding choice or a matchmaking quality for a first mammal in relation to a plurality of different second mammals by comparing genotypes thereof, wherein the arrangement comprises:
an access to a first database comprising genotyping data of known traits and coefficients weighting severity of said traits, such as mutation, disease risk, lethality and/or affected breeds of mammal species of said mammals to be matchmade,
an access to a second database comprising genotyping data for at least certain loci common to each of said mammals to be matchmade,
an access to a group of markers for said loci to be determined for matchmaking, where
i) at least first portion of the markers relates to loci to be compared in said second databases and having the coefficients in various known gene based traits in said first databases for possible breeding combinations derived in said second database, and
ii) at least second portion of said markers used in determining differs from said first portion of said markers,
wherein in determination of value of the match for said first mammal with said plurality of the different second mammals the arrangement is configured to:
determine first results of said coefficients weighting the severities of the traits for each potential breeding pair of the first mammal with each of said plurality of the different second mammals by using said first portion of the markers, and
determine second results relating to a diversity for each potential breeding pair of the first mammal with each of said plurality of the different second mammals by using said second portion of the markers,
determine a total result determining the matchmaking quality for each potential breeding pair by combining said first and second results for each of said potential breeding pair, and
manipulate said total results for each of said potential breeding pairs in order to determine values of matches for said first mammal.

14. The arrangement of claim 13 for determining said matchmaking quality, wherein the arrangement is configured to:
determine first results of said coefficients weighting the severities of the traits for the potential breeding partners of the first mammal with each of said plurality of the different second mammals by using said first portion of the markers, where a sub-result of the first results relates to a certain genotype and has a certain sub-coefficient, whereupon the first results are determined by summing said sub-coefficients of said sub-results, and
determine second results relating to the diversity by using said second portion of the markers so that if genotype of a certain locus is different for said first mammal than for the potential partner of said different second mammals in the second database, said sub-coefficient for said locus is rewarded and if genotype of said locus is same for both mammals, said sub-coefficient for said locus is not awarded or is penalized, whereupon the second results are determined by summing said sub-coefficients, and
determine said total result by summing said first and second results.

15. The arrangement of claim 13 for determining said matchmaking quality, wherein the arrangement is configured to:
provide a group of virtual descendants for different combinations of said first and at least one of said second different mammals to be matchmade, where the arrangement is further configured to:
i) compare genotyping data of the first mammal's genome with corresponding genotyping data of second different mammal's genome in the second database,
ii) determine possible combined genotypes based on said compared genotyping data for each of virtual descendants to be provided,
iii) determine an individual index for health related traits and/or genetic diversity for each of said virtual descendants to determine the value of disease risks of said descendants and breeding value of said two mammals to be matchmade, comprising:
a) comparing the genotyping data of each of said individual virtual descendants' genome corresponding to said first portion of said markers with corresponding genotyping data of said first database in order to determine coefficients of the first results between said determined regions and data of said first database and thereby determine said individual index for each of said individual virtual descendants of a probability or risk or severity of the traits, and
b) combining said first results and second results, such as averages of said first results and second results to determine said individual index of said virtual descendants and breeding value of said two mammals to be matchmade as said total result.

16. A computer program product for determining an advised breeding choice or a matchmaking quality for a first mammal in relation to a plurality of different second mammals by comparing genotypes thereof, characterized in that it comprises program code means stored on a computer-readable medium, which code means are arranged to perform all the steps of the method of claim 1, when the program is run on a computer.
